(19) United States
(12) Patent Application Publication
(10) Pub. No.: US 2025/0010027 A1
(43) Pub. Date: Jan. 9, 2025

(54) Medical Device Assembly

(71) Applicant: Hollister Incorporated, Libertyville, IL (US)

(72) Inventors: Vivienne McNulty, Ballina (IE); Michael G. Murray, Ballina (IE); Shane O'Malley, Ballina (IE); Adam J. Foley, Swords (IE)

(21) Appl. No.: 18/712,379

(22) PCT Filed: Nov. 21, 2022
(86) PCT No.: PCT/US2022/050608

§ 371 Date: May 22, 2024

Related U.S. Application Data
(60) Provisional application No. 63/284,271, filed on Nov. 30, 2021.

(51) Int. Cl. A61M 25/00

(52) U.S. Cl. CPC A61M 25/002; A61M 25/0017; A61M 2205/0222; A61M 2209/06; A61M 2210/1089

(57) ABSTRACT

Medical device assemblies (100, 300) are disclosed. The medical device assemblies include a body-insertable medical device (124) and a case (102, 302) for holding or storing the medical device before (and after) use, and a cap (110, 310) for sealing the device within the case. The assembly further includes a hydrating liquid (130) that contacts a portion of the medical device. The medical assembly is configured to allow drainage of residual liquid away from the cap and reduce the likelihood of spillage of the hydrating liquid upon opening of the case.

### CROSS-REFERENCE TO RELATED APPLICATION

This application claims the benefit of U.S. Provisional Application Ser. No. 63/284,271, filed Nov. 30, 2021, the disclosure of which is hereby incorporated by reference in its entirety.

### FIELD OF THE DISCLOSURE

The present disclosure generally relates to medical device assemblies that include a case and a medical device stored within the case. More particularly, the present disclosure relates to medical device assemblies having a case configured to hold a hydration fluid and a hydratable medical device having a body-insertable member and a drainage member, wherein the hydratable medical device is configured to be removably enclosed by the case. In an example, the hydratable medical device may be a hydrophilic intermittent urinary catheter. Even more particularly, the present disclosure relates to medical device assemblies that include a case configured to hold a medical device and a hydration fluid whereby spillage of the hydration fluid upon opening of the case is minimized.

Certain medical devices, such as intermittent urinary catheters are commonly used by those who suffer from various abnormalities of the urinary system, such as urinary incontinence. Urinary catheters typically include a polymeric shaft portion that is inserted into a user's urethra and a drainage member/funnel that is attached to the shaft distal end and is used to help facilitate and drain urine and provide a gripping member for the user. The catheter polymeric shaft may have a hydrophilic coating to ease insertion and advancement of the shaft into the urethra. As such, the catheter is typically hydrated or otherwise lubricated with a hydration/lubricating medium prior to insertion. Where the hydration medium is a liquid, the packages that house the catheters prior to use may include a small volume of a hydrating liquid that contacts the catheter shaft during storage.

One potential challenge of providing a catheter package that includes a hydrating liquid is avoiding or at least minimizing spillage of the hydrating liquid upon opening of the package. Inasmuch as many of the intermittent catheter packages are compact that can be discreetly transported by the user (such as in a purse or handbag), the liquid inside these catheter packages may flow into parts of the catheter package at or near the point of opening. Consequently, hydrating liquid may spill from the case when the case is opened.

Thus, some catheter packages are provided with inserts that prevent or at least minimize the liquid inside the package from spilling upon opening of the package. For example, WO 2017/185052 and WO 2018/156589 describe medical device packages that include a case in the form of a hollow tube and a hinged flip cap. The hollow tube receives the medical device e.g., catheter, and contains a hydrating liquid. The case includes an insert (described as a truncated “liner”) that serves as a plug to retain hydration liquid and reduces the risk of spillage. The contents of WO 2017/185052 and WO 2018/156589 are incorporated herein by reference.

Whether or not the catheter package includes a plug of the type described in the above referenced publications, there remains the possibility that the hydrating liquid may enter the catheter shaft (through the eyelets) and reside in the cap which can also result in spillage upon opening of the package. Thus, it would be desirable to provide a catheter package that prevents residual hydrating fluid from residing in the cap or at or near the opening. The medical device assemblies as shown and described below address this need.

### SUMMARY

There are several aspects of the present subject matter which may be embodied separately or together in the devices and systems described and claimed below. These aspects may be employed alone or in combination with other aspects of the subject matter described herein, and the description of these aspects together is not intended to preclude the use of these aspects separately or the claiming of such aspects separately or in different combinations as set forth in the claims appended hereto.

In one aspect, a medical device assembly is disclosed. The medical device assembly includes a case having an elongated, generally cylindrical body that is closed at a first end and open at a second end. A cap is hingedly attached to the second end and is selectively movable between an open position and a closed position. The body has a distal portion and a proximal portion and an interior surface and an exterior surface. The interior surface defines a chamber configured to contain a hydrating liquid and includes a plurality of ribs in the proximal portion. The ribs extend from the interior surface of the body and define fluid channels between the ribs. The medical device assembly also includes a medical device configured to be removably contained within the case. The medical device has a distal drainage member that includes at least one drainage slot and a proximal tube extending from the drainage member. The at least one drainage slot of the drainage member is in fluid communication with the fluid channels.

In another aspect, a case for holding a medical device is disclosed. The case includes an elongated generally cylindrical body which is closed at a first end and open at a second end. The body includes an interior surface and an exterior surface. The interior surface defines a chamber configured to hold at least a portion of the medical device. The exterior surface defines a chamfered region near the closed first end. A cap is hingedly connected to the second end, and is selectively movable between an open position, wherein access is provided to the second end and a closed position, wherein the cap prevents access to the second end.

In a further aspect, a package for holding a medical device is disclosed. The package includes a case having an elongated generally cylindrical body which is closed at a first end and open at a second end. A cap is hingedly connected to the second end, and is selectively movable between an open position, and a closed position. The body has a distal portion and a proximal portion and an interior surface and an exterior surface. The interior surface defines a chamber configured to contain a hydrating liquid and includes a plurality of ribs in the proximal portion. The ribs inwardly extend from the interior surface of the body into the chamber and define fluid channels between the ribs.

### BRIEF DESCRIPTION OF THE DRAWINGS

FIG. 1 is a side view of an embodiment of a medical device assembly with the case in a closed position;

FIG. 2 is a side cross-sectional view of a case of the medical device assembly of FIG. 1, showing the case in an open position;

FIG. 3 is a side view of the case of FIG. 2, showing the case in the open position, laying horizontally, and contacting a surface.

FIG. 4 is a cross sectional, perspective view of the medical device assembly of FIG. 1, showing a medical device positioned within the case.

FIG. 5 is a top cross-sectional view of the medical device assembly of FIG. 4, showing the case interior containing a medical device and a hydration fluid.

FIG. 6 is a bottom cross-sectional view of the medical device assembly of FIG. 4, showing the case interior containing a medical device.

FIG. 7 is a bottom cross-sectional view of a second embodiment of a medical device assembly, showing a case interior containing a medical device.

FIG. 8 is an enlarged plan view of the medical device of FIG. 4, showing the medical device drainage member.

FIG. 9 is a side cross-sectional view of another embodiment of a case of a medical device assembly, showing the case including a grommet.

FIG. 10 is an enlarged view of the case of FIG. 9, showing the grommet within the case.

FIG. 11 is a cross-sectional, perspective view of the medical device assembly of FIG. 9 in an open position, showing a medical device positioned within the case interior.

FIG. 12 is a partial-cross sectional view of the medical device assembly of FIG. 9, in an inverted orientation; and

FIG. 13 is a perspective view of the grommet of FIG. 9, shown outside of the case.

### DESCRIPTION OF THE ILLUSTRATED EMBODIMENTS

The embodiments disclosed herein are for the purpose of providing a description of the present subject matter, and it is understood that the subject matter may be embodied in various other forms and combinations not shown in detail. Therefore, specific embodiments and features disclosed herein are not to be interpreted as limiting the subject matter as defined in the accompanying claims.

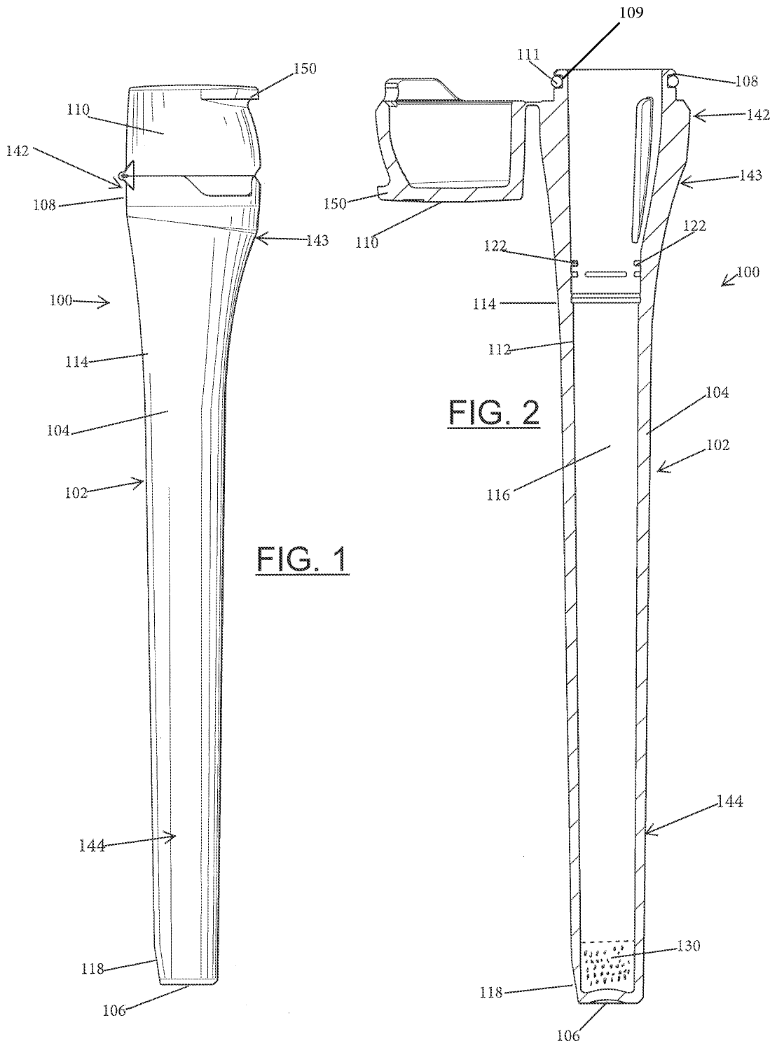

Catheter assemblies according to the present disclosure and their individual components may be variously configured without departing from the scope of the present disclosure, but in one embodiment, an intermittent urinary catheter assembly is configured as shown in FIG. 1.

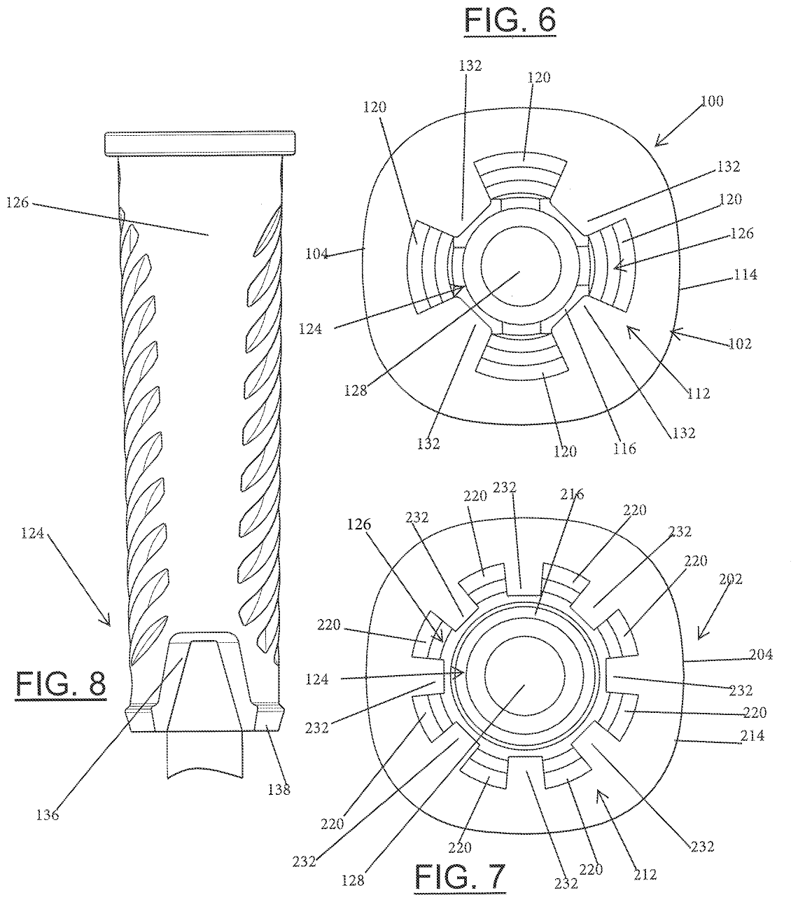

FIGS. 1-6 show a medical device assembly 100. As shown in FIGS. 1-2, the assembly 100 includes a package including a case 102 having an elongated, generally cylindrical body 104 that is closed at a first end 106 and openable at a second end 108. Case 102 provides a compact package for the medical device (e.g., catheter) and may have a length of approximately 136 mm.

A cap 110 is hingedly attached to the second end 108 of case 102 and is selectively movable between an open position and a closed position. The case 102 is shown in the closed position in FIG. 1 and the open position in FIG. 2. When in the open position access is provided to the second end 108 and when in the closed position the cap 110 prevents access to the second end. In one embodiment, cap 110 and body 104 may be come together in a snap fit. The cap 110 may include a lip 150 to facilitate opening of the case 102. In use, a user may position a finger under the lip 150 and push against the lip 150 in a direction away from the second end and the cap 110 is rotated along the hinge, exposing the interior of the case. When closed, the cap 110 and body 104 form a liquid tight seal. In that regard, case 102 may include a groove 109 that receives an O-ring 111 which engages an interior surface of cap 110. The cap 110, which may be a generally cup-shaped shell sized and/or otherwise configured to accommodate an end of a medical device, such as a portion of a catheter funnel when in the closed position. Further aspects of the cap and its engagement with the case are described in WO 2017/185052 and WO 2018/156589, both of which are incorporated by reference.

As shown in FIGS. 1-2, the body 104 of case 102 has a distal portion 142, a proximal portion 144, and a transitional neck portion 143 between the distal and proximal portions. Body 104 of case 102 further includes an interior surface 112 and an exterior surface 114. The terms “distal” and “proximal” are used throughout this disclosure. When used in the context of the catheter or the catheter tube that is inserted into the body of the user, the term “proximal” is used to refer to that end or portion of the catheter tube that during use is closer in proximity to the user's body and/or initially enters the user's body upon insertion. The term “distal” is used to refer to an end or portion of the catheter tube that is opposite the proximal end or portion and is typically further away from the user's body. For the sake of consistency, when the terms “distal” and “proximal” are used in the context of a package or case that receives or houses the catheter or catheter tube but are not intended for introduction into the user's body, a proximal end or proximal portion is that end or portion closer to the proximal end of the catheter tube when the catheter tube is housed or carried by such package or housing, while the distal end or portion is located opposite to such proximal end or portion.

FIG. 1 shows the exterior surface and FIG. 2 shows the interior surface. The exterior surface 114 may be made of any appropriate material including a polymeric material such as polyethylene terephthalate (PET). The exterior surface 114 may be rigid in order to protect the contents of the case 102. In the embodiment shown, case 102 may have a generally cylindrical cross-sectional shape. In an alternative embodiment, the exterior surface may have a generally rectangular shape. The case 102 may have a variable outer diameter. For example, as seen in FIGS. 1 and 2, distal portion 142 of case 102 flares outwardly at neck portion 143. Additionally, in further alternative embodiments, the exterior surface may take any other known appropriate general shapes. As noted above, additional features of the case 102, cap 110 are described in WO 2017/185052 and WO 2018/156589, both of which are incorporated by reference in their entireties.

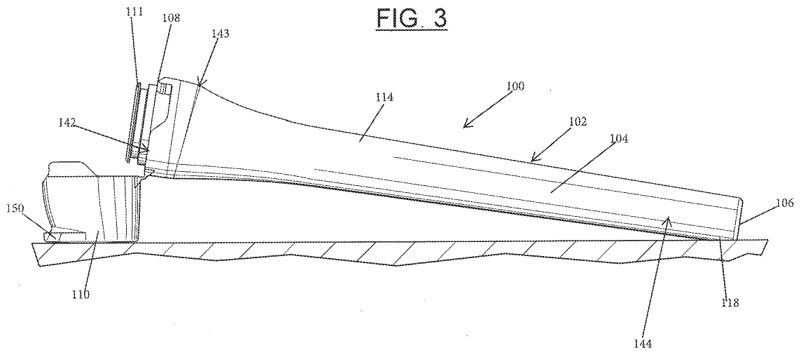

As shown in FIG. 3, the exterior surface 114, defines a chamfered area 118. The chamfered area 118 may be a flat seat positioned adjacent to the first end 106. The chamfered area 118 may define an angle α of approximately 5-30 degrees, or more preferably 5-20 degrees, or even more preferably 6-12 degrees relative to the tangent of exterior surface of case 102 (see FIGS. 1 and 3). In a more particular embodiment, the chamfered area may define an angle α of approximately 9 degrees. As shown, when the case 102 is laid on a surface for example, after removal of the medical device for use, the chamfered area 118 rests against the surface near the first end 106, while the top of the open cap 110 rests against the surface near the second end 108, causing the body 104 to incline such that the distal portion 142 is elevated. This inclined position helps increase stability when the case 102 is open and in the horizontal position and helps to prevent the case from inverting and the case 102 liquid contents from spilling.

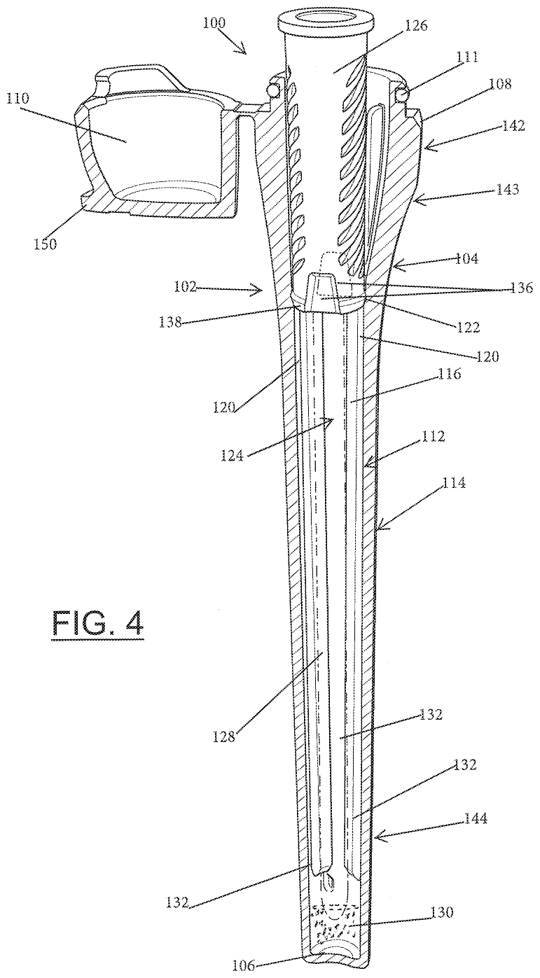

As shown, in FIG. 2, the interior surface 112 defines a chamber 116 configured to contain a hydrating liquid 130. In one embodiment, the hydrating liquid 130 may be water. As shown in FIGS. 4-6, the interior surface 112 includes a plurality of ribs 132 in the proximal portion 144. The ribs 132 extend inwardly toward chamber 116 from the interior surface 112 of the body 104. The spaces between the ribs 132 define fluid channels 120. The ribs 132 may include a leading edge that tapers toward the interior surface 112 as the ribs 132 extend in a proximal direction. These fluid channels 120 allow liquid which may accumulate in the cap 110 and/or distal portion 142 during storage or transportation, to flow back into the proximal portion 144 of the chamber 116 when case 102 is placed in an upright position such as during opening of the cap 110.

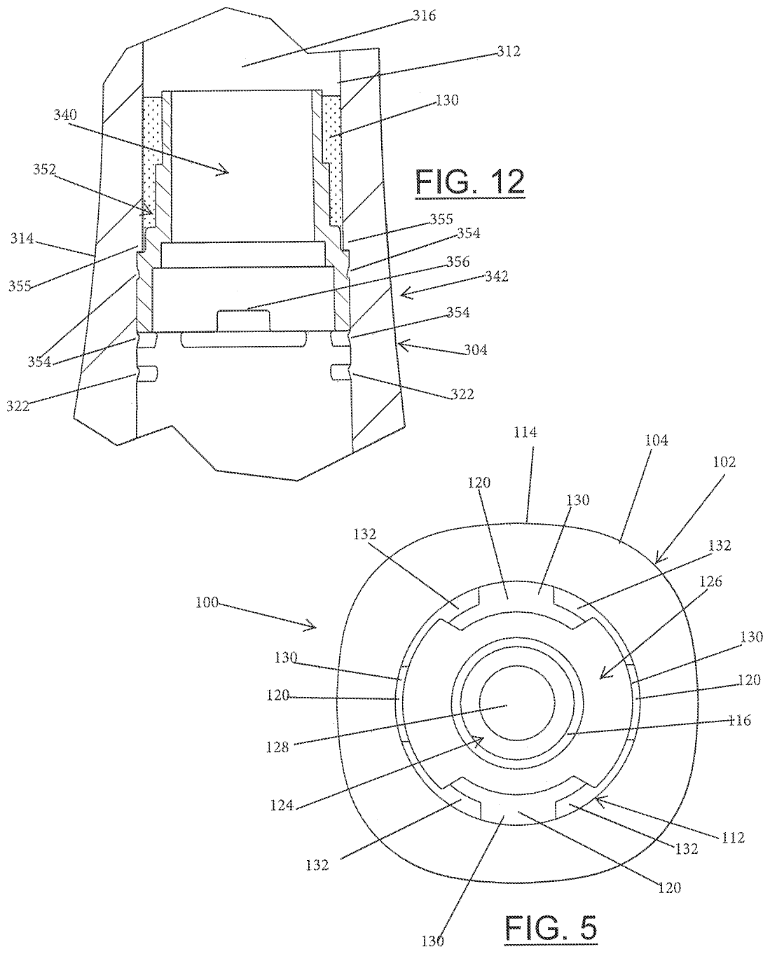

As further shown in FIGS. 4-6, the assembly 100 includes a removable medical device 124. At least a portion of the medical device 124 is configured to fit within the chamber 116. As shown in FIG. 4, the medical device 124 has a distal drainage member 126 and a proximal (catheter) tube 128 extending from the proximal end of the drainage member 126. The drainage member 126 includes at least one drainage slot 136 (best shown in FIG. 8), which may be a recess in the outer surface of drainage member 126 at or near the proximal end of the drainage member. The drainage slot 136 of the drainage member 126 is in fluid communication with the fluid channels 120. Hydrating liquid that flows into the cap 110 and/or distal portion 142 during transportation and storage may flow into the at least one drainage slot 136 and into the channels 120 of the interior surface 112. When the medical device 124 is inserted into the case 102 the drainage member 126 is positioned such that the slots 136 are aligned with the channels 120 ensuring fluid communication between the distal 142 and proximal 144 portions of the case 102. The ribs 132 defining the channels 120 (FIG. 5) may help to hold the medical device 124 in proper position.

FIGS. 4-6 show an embodiment of a case 102 with the interior surface 112 having four ribs 132. As such four channels 120 are formed by the ribs 132 that align with the slots 136 of the medical device 124.

FIG. 7 shows an alternate embodiment of a case 202 having a body 204 having an interior surface 212 and an exterior surface 214. The interior surface 212 defines a chamber 216 and includes eight ribs 232 and forming eight channels 220. In other embodiments, any suitable number of ribs and channels and may be used. As a corollary, in other embodiments, the drainage member 126 may include a plurality of slots corresponding to the number of, and fluidly communicating with, the given plurality of channels.

As shown in FIGS. 2 and 4, the interior surface 112 includes a step 122 for retaining drainage member 126. As shown in FIG. 8, the drainage member 126 may include a flange or engagement member 138 configured to engage step 122. In one particular embodiment, the drainage member 126 shown in FIG. 8 is a funnel including a lower flange 138, the flange 138 being configured to engage the interior surface 112 of the case 102. The step 122 provides a docking such that as the medical device 124 is inserted into the case 102, the drainage member flange 138 is eventually pressed past step 122, docking the medical device 124 and ensuring that the medical device 124 remains in proper position while stored and transported. Though the drainage member 126 is a funnel, other known appropriate drainage members may be used in alternative embodiments.

The medical device 124 shown in FIGS. 4-6 is preferably a hydrophilic urinary catheter. The hydrophilic urinary catheter includes the funnel 126 at a distal portion of the catheter and the tube 128 at a proximal portion. The tube 128 may be held in the chamber 116 such that the hydrating liquid 130 comes into contact with the catheter tube 128. The urinary catheter may also include a hydrophilic coating configured to be hydrated by the hydrating liquid 130, such that when the catheter is removed from the case 102, the catheter surface is lubricious and ready for use. Though a urinary catheter is used in these embodiments, alternative embodiments may contain any appropriate medical device including hydratable medical devices.

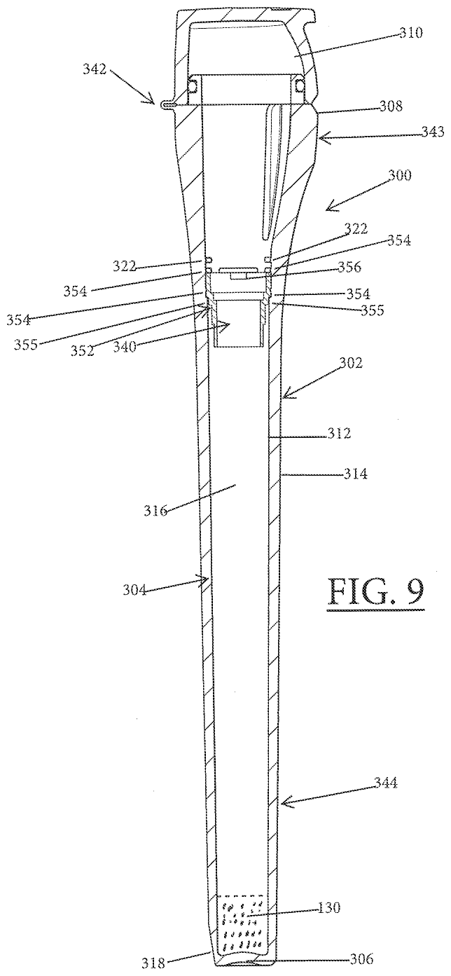

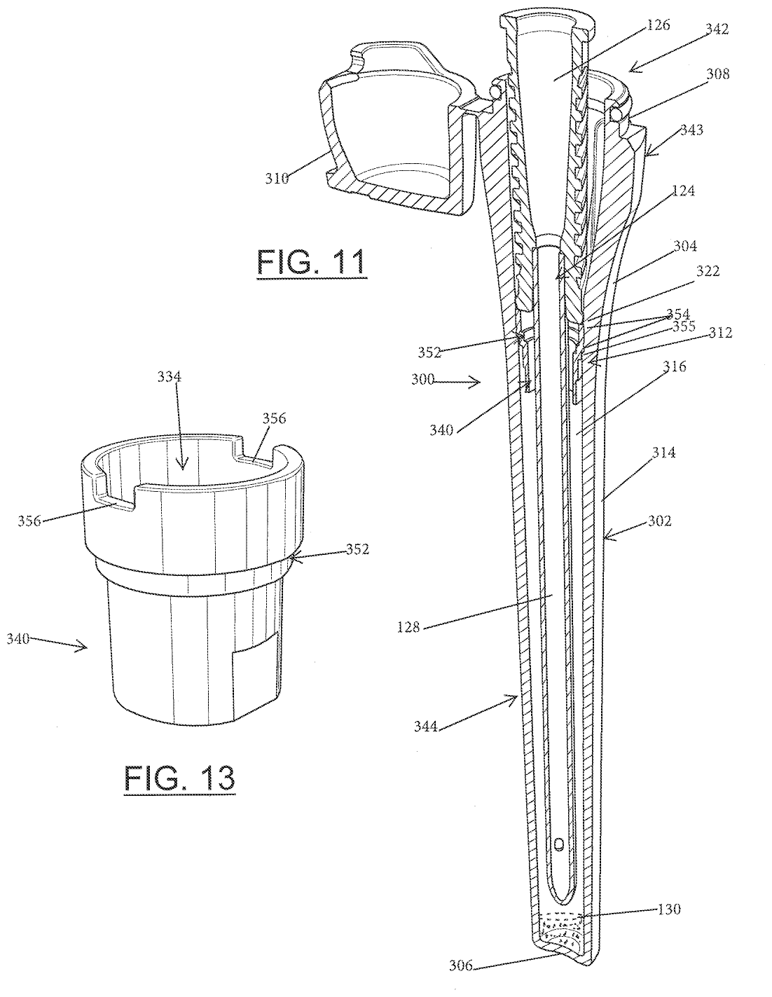

FIGS. 9-13 show an alternative embodiment of a medical device assembly 300. As shown in FIGS. 9-12, the assembly 300 includes a case 302 for containing the medical device 124, similar to the case 102 shown in FIGS. 1-8, described above. The case 302 is closed at a first end 306 and open at a second end 308. As shown in FIGS. 9 and 11, the case includes a body 304 having a distal portion 342, a neck portion 343, a proximal portion 344, an interior surface 312 and an exterior surface 314. The exterior surface 314 may include a chamfered area 318 near the first end 306. The interior surface 312 defines a chamber 316 configured to contain the hydrating liquid 130 and the medical device. A cap 310 is hingedly attached to the body 304 and opens and closes like the cap 110 of the case 102. The interior surface 312 may further include one or more beads 322 configured to receive the drainage member 126 for docking and the tube 128 is contained by the chamber 316.

Case 302 of FIGS. 9-12 includes an insertable grommet 340 such as the plug described in WO 2017/185052 and WO 2018/156589, incorporated by reference herein. FIGS. 9, 10, and 12 show the case 302 including the insertable grommet 340, without the medical device 124 inserted. FIG. 11 shows the case 302 with the medical device 124 inserted.

The grommet 340 is inserted into the distal portion 342 of the case 302 and includes one or more notches 356 which may be included for ease of manufacturing the grommet 340. The grommet 340 by itself is shown in FIG. 13 and includes two notches 356. As shown most clearly in FIG. 13, the grommet 340 is generally cylindrical and has a lumen 334 configured to allow passage of at least a portion of the medical device 124, for example the (catheter) tube 128, through the grommet 340 and into the chamber 316.

The grommet 340 is configured to prevent the hydrating liquid 130 from flowing out of the case 302 when the case 302 is inverted, in order to minimize fluid from collecting in the distal portion or near the opening or prevent spillage of the liquid 130 from the case 302 when the case is opened. FIG. 12 shows a portion of the case 302 inverted and shows that the grommet 340 prevents the liquid 130 from flowing toward the case second end 308. As shown in FIGS. 9-13, the grommet 340 includes a tiered profile 352 wherein different tiers of grommet 340 may have a different outer diameter. For example, one tier of grommet 340 may have an outer diameter that substantially corresponds to the inner diameter of case 302, allowing grommet 340 to be press-fit into case 302. Other (lower) tiers of grommet 340 may have outer diameters that are smaller than the inner diameter of the case interior surface, thereby resulting in a gap between the outer surfaces of the lower tiers and the case interior. Such gap(s) between the grommet 340 and the interior wall of case 302 provide liquid retention regions as best seen in FIG. 12. The grommet may be separately molded and made of any appropriate material including polymers such as polyethylene terephthalate (PET).

As shown in FIGS. 9-12, the interior surface 312 of case 302 may further include one or more beads 322, 354 configured to contact at least a portion of the drainage member 126 and grommet 340. Beads 322, 354 may define inwardly extending protrusions on the interior surface 312 of case 302. Upper bead 322 may be provided to contact and loosely retain at least a portion of the drainage member 126 (such as lower flange 138 shown in FIG. 8) of an inserted catheter. Beads 354 may be provided to contact and retain one or more portions of the grommet 340 and hold the grommet 340 securely in place after insertion.

As shown in FIGS. 9-12 the interior surface 312 includes a plurality of (lower) beads 354. For example, the interior surface 312 may include two beads 354, as shown. The beads 322, 354 may be continuous about the interior circumference of case 302 or, as shown in FIGS. 2 and 9, beads 322, 354 may be discontinuous to define a plurality of interrupted bead segments.

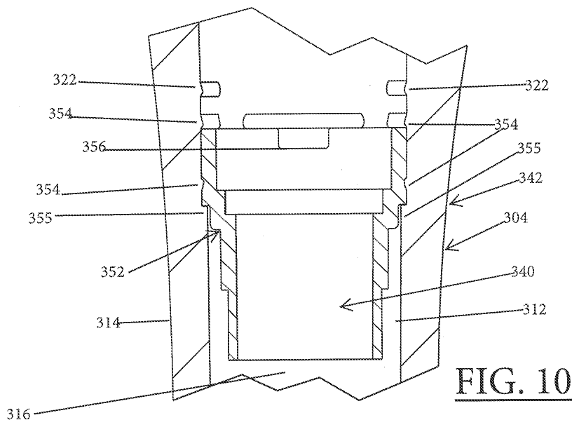

Additionally, the interior surface 312 may include a shelf 355 configured to receive a portion or tier of the grommet 340. The shelf 355 may extend inwardly and perpendicularly from the interior surface 312. As shown in FIG. 10, the shelf 355 is defined by a portion of the interior surface 312 having a smaller inner diameter than the interior surface 312 distal to said shelf 355. The shelf 355 is positioned at a location on the interior surface 312 that is proximal from the at least one bead 354.

In an alternative embodiment (not shown), the grommet may be inserted into cases 102 and 202 as shown in FIGS. 1-7 and described above. In this alternative embodiment, the grommet may help reduce fluid flow to the distal portion of the case in order to minimize fluid, that is not in the channels, from collecting in the distal portion or near the opening when the case is inverted, and to reduce potential spillage of the liquid from the case when the case is opened.

It will be understood that the embodiments described above are illustrative of some of the applications of the principles of the present subject matter. Numerous modifications may be made by those skilled in the art without departing from the spirit and scope of the claimed subject matter, including those combinations of features that are individually disclosed or claimed herein. For these reasons, the scope hereof is not limited to the above description but is as set forth in the following claims, and it is understood that claims may be directed to the features hereof, including as combinations of features that are individually disclosed or claimed herein.

What is claimed is:

1. A medical device assembly, comprising:
a case having an elongated, generally cylindrical body which is closed at a first end and open at a second end and a cap hingedly attached to the second end, the cap being selectively movable between an open position and a closed position, wherein the body comprises a distal portion and a proximal portion and an interior surface and an exterior surface, the interior surface defining a chamber and including a plurality of ribs in at least said proximal portion, said ribs extending from said interior surface of said body and defining fluid channels between said ribs; and
a medical device removably contained within the case, the medical device having a distal drainage member and a proximal tube extending from said drainage member, the drainage member including at least one drainage slot, wherein at least a portion of the medical device is configured to fit within the chamber;
wherein said at least one drainage slot in said drainage member is in fluid communication with said fluid channels; and
a hydrating liquid.

2. The assembly of claim 1 wherein said interior surface comprises a step for receiving the drainage member.

3. The assembly of claim 1, wherein the drainage member includes a plurality of slots configured to fluidly communicate with said channels.

4. The assembly of claim 2, wherein the drainage member comprises a flange for engagement with said step.

5. The assembly of claim 1, wherein the case further includes an insertable grommet.

6. The assembly of claim 1, further including a chamfered area is a flat seat positioned adjacent to the first end.

7. The assembly of claim 1, wherein the drainage member is a funnel including a lower flange, the flange being configured to removably engage said interior surface of said case.

8. The assembly of claim 1, wherein the medical device is a hydrophilic urinary catheter.

9. The assembly of claim 1, wherein the hydration liquid is water.

10. A case for holding a medical device, comprising:
an elongated generally cylindrical body which is closed at a first end and open at a second end, wherein the body comprises an interior surface and an exterior surface, the interior surface defining a chamber configured to hold at least a portion of the medical device, and the exterior surface including a chamfered region near said closed first end; and
a cap hingedly connected to the second end, the cap being selectively movable between an open position, wherein access is provided to the second end and a closed position, wherein the cap prevents access to the second end.

11. The case of claim 10 comprising a liquid hydrating medium in said chamber.

12. The case of claim 10, wherein said chamfered region defines an angle α of 9.20 degrees relative to the tangent of the case outer surface.

13. A package for holding a medical device comprising:
an elongated, generally cylindrical body which is closed at a first end and open at a second end and a cap hingedly attached to the second end, the cap being selectively movable between an open position and a closed position, wherein the body comprises a distal portion and a proximal portion and an interior surface and an exterior surface, the interior surface defining a chamber and including a plurality of ribs in said proximal portion, said ribs inwardly extending from said interior surface of said body into said chamber, and defining fluid channels between said ribs.

14. The package of claim 13 wherein said package has a variable outer diameter.

15. The package of claim 13 wherein said package comprises an exterior surface that has a generally cylindrical cross-sectional shape.

16. The package of claim 13 wherein said package comprises an exterior surface that has a generally rectangular cross-section.

17. The package of claim 13 wherein said body distal portion flares outwardly.

18. The package of claim 13 said ribs comprise a leading edge that tapers toward said interior surface as said ribs extend in a proximal direction.

19. The package of claim 13 comprising a hinged cap.

20. The package of claim 13 comprising a docking step in the interior surface of said body.
